(19) United States
(12) Patent Application Publication
(10) Pub. No.: US 2019/0125260 A1
(43) Pub. Date: May 2, 2019

(54) METHOD OF JOINING ELASTOMERIC LIPS,PRESS-SWITCH SYSTEM, AND WEARABLE MONITOR

(71) Applicant: ICENTIA INC., Québec (CA)

(72) Inventors: David LEVESQUE, St-Augustin-de-Desmaures (CA); Yannick LE DEVEHAT, Québec (CA)

(21) Appl. No.: 16/093,153

(22) PCT Filed: Apr. 12, 2016
(86) PCT No.: PCT/CA2016/050420

§ 371 Date: Oct. 12, 2018

(51) Int. Cl. A61B 5/00; B29C 70/84

(52) U.S. Cl. CPC A61B 5/6801; B29L 2031/752; B29C 70/845

(57) ABSTRACT

A housing can have two superposed housing members and a joint member connecting lips of the housing members to one another along at least a portion of a periphery. The housing members and the joint member can all be made of an elastomeric material, and thus be flexible, compressible, and have elasticity. An electronic unit within an air-filled cavity between the housing members can be received on a board, itself being received on one of the housing members, and having a press-switch unit covered by the other one of the housing members and forming part of a press-switch system.

### FIELD

The improvements generally relate to the field of elastomeric housings for electronics.

### BACKGROUND

At least some electronic devices require electronics to be housed in a housing made with elastomeric materials. This can be for providing flexibility/bending ability to the housing, water-resistance to the electronics contained therein, or both, for instance. Such devices can be subject to manufacturing cost and ease-of-use design considerations for instance.

Although such housings have been satisfactory to a certain degree, there remains room for improvement in areas such as manufacturing methods and press-switches covered by an elastomeric material.

### SUMMARY

Concerning the manufacturing aspects, it was attempted to provide the housing in two generally sheet-like housing members of elastomeric material. Such components can be referred to as the first housing member and the second housing member, and can be provided with an internal cavity to receive an electronic unit, for instance. The edges of the first housing member and the second housing member being superposed to one another when in the assembled configuration, and can be referred to herein as adjacent lips. It was attempted to join the lips by overmoulding a third joint member of a thermoformable elastomeric material in a manner to cover the ends of the lips and partially overlap the lips on each face. However due to the surface tension of the thermoformable elastomeric material, the pressure of the closing mould forces the adjacent lips away from each other towards the inner surface of the mould. Then at the injection pressures, the thermoformable elastomeric material, then in a relatively liquid/viscous state, tended to penetrate between the two lips and further push the two lips against the mould, preventing a satisfactory amount of material from overlapping over the faces of the lips.

A satisfactory attempt was achieved by using a mould with alternating abutments and rib negatives. The abutments pressed against and sandwiched the two lips at regular intervals along their length. The distance between the abutments was selected in a manner that it was sufficiently small to limit the degree of freedom of the sheet edges within the rib negatives (where the lips are not compressed by the abutments), in a manner that while a certain amount of the seal material could penetrate between the edges within the rib negatives, a satisfactory amount of seal material nonetheless overlapped the edges on each face and formed the ribs which can satisfactorily join and seal the lips to one another once formed. The resulting seal structure can have a flange extending from ends of the jointed lips, and running along the length of the joined lips, and plurality of pairs of ribs, each pair having two opposed ribs protruding from the flange and extending partially over a corresponding one of the lip faces. The pairs of ribs being interspaced from one another along the length of the lips. This construction can retain a satisfactory level of transversal flexibility for some applications.

Accordingly, in accordance with one aspect, there is provided an electronic device comprising an electronic unit housed within a housing, said housing comprising two housing members and a joint member each being made of an elastomeric material, the two housing members having superposed lips extending at least partially along a periphery thereof, the joint member having a flange projecting from ends of the lips, and a plurality of pairs of ribs, the pairs of ribs being interspaced from one another along the superposed lips, and each rib of each pair protruding from the flange and extending over a face of a corresponding one of the lips.

In accordance with another aspect, there is provided a method of joining lips of an elastomeric material, the method comprising: positioning the two lips in a superposed manner in a mould, the mould compressing the superposed lips in a plurality of abutments interspaced from one another along the length of the lips, the plurality of abutments being interspaced from one another by corresponding rib negatives; overmoulding a joint member onto the superposed lips in the mould, the joint having a flange projecting from ends of the lips, and a plurality of pairs of ribs filling the rib negatives, each rib of a corresponding pair protruding from the flange and extending over a face of a corresponding one of the lips.

In accordance with another aspect, there is provided: a wearable monitor having a flexible housing having two opposite ends each bearing a corresponding electrode connector, and a monitoring unit received within the housing and connected to both electrode connectors, said housing comprising two housing members and a joint member each being made of an elastomeric material, the two housing members having superposed lips extending at least partially along a periphery thereof, the joint member having a flange projecting from ends of the lips, and a plurality of pairs of ribs, the pairs of ribs being interspaced from one another along the superposed lips, and each rib of each pair protruding from the flange and extending over a face of a corresponding one of the lips.

In another aspect, it can be desired to provide a press-switch system by which a user can trigger a press-switch unit made integral to an electronic unit covered by housing member of elastomeric material. To this end, a press-switch system can be provided with an outer depression in the elastomeric material above the switch unit to allow the user to easily locate the switch's location and can also impede inadvertent activation of the switch by the user. Moreover, a ‘plate’ of relatively rigid material can be held in place between the elastomeric material and the switch unit, which can help both i) in allowing efficient activation of the press-switch when the patient applies pressure by a finger or thumb on the elastomeric material (as compared to a scenario where the material in contact with the switch unit would be the soft elastomeric material), and ii) in receiving the tactile feedback from the switch in the form of a tactile ‘click’ feeling via the plate. It was found that this could significantly improve the user experience by contrast with a scenario in which the elastomeric material directly covers the switch unit. Indeed, when the elastomeric material directly abutted the switch unit, its elasticity limited efficiency of activation of the switch unit, and limited the tactile feedback received by the user.

Accordingly, in accordance with this latter aspect, there is provided a wearable monitor having a flexible housing having two opposite ends each bearing a corresponding electrode connector, the housing having two housing members joined to one another and made of an elastomeric material, a monitoring unit received between the housing members and connected to both electrode connectors, a press-switch unit connected to the monitoring unit and covered by one of the housing members, the press-switch unit having an actuator resiliently movable along a pressing axis to activate the press-switch unit, a plate held between the member of elastomeric material and the actuator, wherein the press-switch unit can be activated by applying pressure along the pressing axis, on an outer face of the housing member covering the press-switch, said pressure being transmitted to the actuator via the plate.

In accordance with another aspect, there is provided a press-switch system for an electronic unit covered by a member of an elastomeric material, the press switch system comprising a press-switch unit connected to the electronic unit, the press-switch unit having an actuator resiliently movable along a pressing axis to activate the press-switch unit, a plate held between the member of elastomeric material and the actuator, wherein the press-switch unit can be activated by applying pressure along the pressing axis, on an outer face of the elastomeric material, said pressure being transmitted to the actuator via the plate. The press-switch system can be embodied in an application other than a wearable monitor, and can be provided with any one or more of the features presented herein in association with a wearable monitor application.

Many further features and combinations thereof concerning the present improvements will appear to those skilled in the art following a reading of the instant disclosure.

### DESCRIPTION OF THE FIGURES

In the figures,

FIG. 1A is an oblique view, showing a portion of an electronic device having two housing members and a joint member;

FIG. 1B is an oblique view of a portion of an electronic device having a press-switch system;

FIG. 2 is an oblique view showing a half of the mould used in overmoulding the joint member to the two housing members in FIG. 1;

FIG. 3 is a cross-sectional view, fragmented, taken along lines 3-3 of FIG. 8;

FIG. 4 is a cross-sectional view, fragmented, taken along lines 4-4 of FIG. 8;

FIG. 5 shows a wearable monitor applied to a patient via medical electrode assemblies;

FIG. 6 is an oblique, exploded view, showing a wearable monitor and medical electrode assemblies of FIG. 5;

FIG. 7 is an oblique, exploded view of the wearable monitor of FIG. 5;

FIG. 8 is a plan view of a mould assembly used in the manufacture of the wearable monitor of FIG. 5; and

FIG. 9 is an oblique view of an outer member of the wearable monitor of FIG. 5.

### DETAILED DESCRIPTION

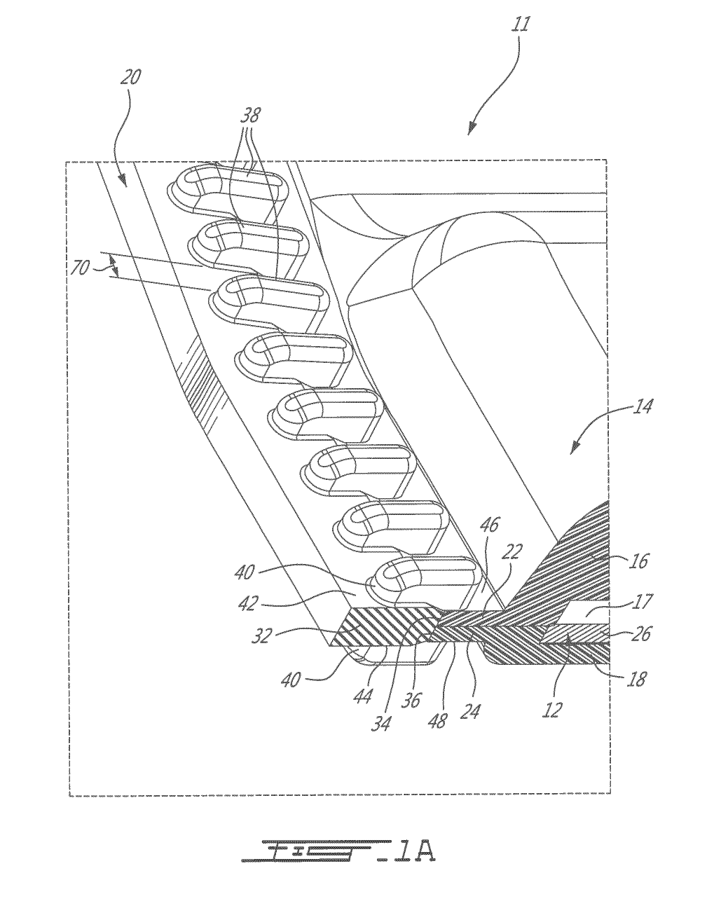

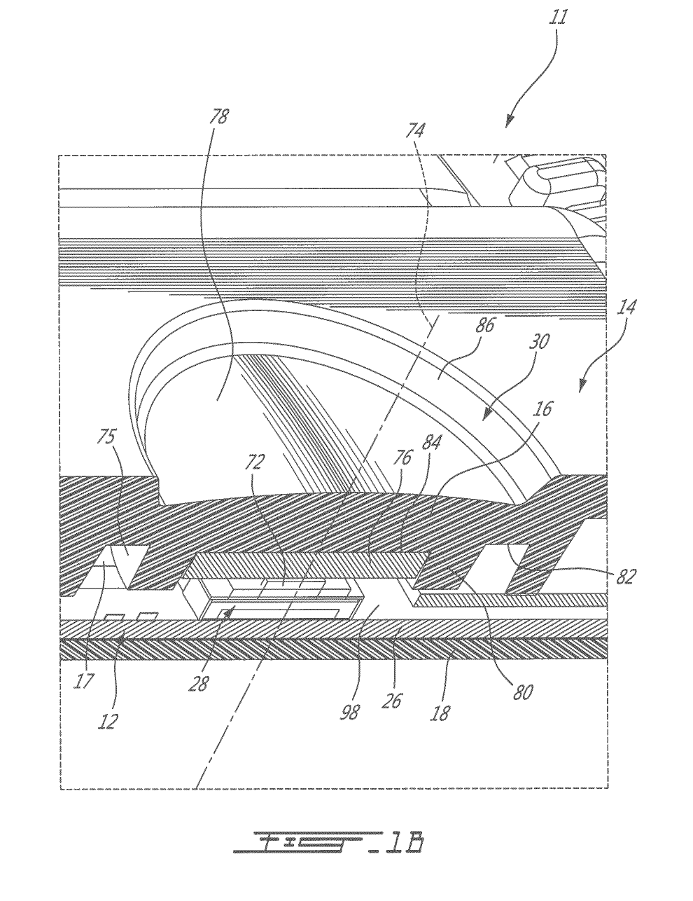

FIGS. 1A and 1B each show a portion of an example of an electronic device 11 having an electronic unit 12 housed within a housing 14. The housing 14 has two superposed housing members 16, 18 and a joint member 20 connecting edges, or lips 22, 24, of the housing members 16, 18 to one another along at least a portion of a periphery of the housing members 16, 18. The housing members 16, 18 and the joint member 20 are all made of an elastomeric material, and are thus flexible, compressible, and have elasticity. In this example, the electronic unit 12 is received on a board 26 (better seen in FIG. 1B), itself being received on one of the housing members 18, and has a press-switch unit 28 which is covered by the other one of the housing members 16 and forms part of a press-switch system 30. The electronic unit 12 is in a sealed cavity 17 between the housing members 16, 18, the sealed cavity 17 is filled with air. Indeed, the presence of electronic components such as a press-switch unit 28 for instance can make direct overmoulding unadapted to the application. Both the joint member 20 and the press-switch system 30 will now be individually and generally described one after the other, prior to providing the details of an example embodiment provided in the form of a wearable monitor 10.

Concerning the joint member 20, it can be seen in FIG. 1A that the joint member 20 has a flange 32 which extends or projects from the adjacent ends 34, 36 of the superposed lips 22, 24. The flange 32 follows the lips 22, 24 continuously along at least a portion of the periphery, and can be said to ‘run’ along at least a portion of the ends 34, 36 of the lips 22, 24 in an orientation which can be qualified as ‘longitudinal’. The joint member 20 further has a plurality of ribs 38. The ribs 38 are arranged in a plurality of opposed pairs 40 which are interspaced from one another along the periphery. Each pair 40 has two opposed ribs 38, with each rib 38 of the pair 40 i) protruding from the flange 32 on a corresponding, opposite side, ii) extending partially along the corresponding face 42, 44 of the flange, and iii) extending partially along the face 46, 48 of the corresponding lip 22, 24. As will now be explained, the joint member 20, including the flange 32 and ribs 38, can be overmoulded to the lips 22, 24 of the housing members 16, 18 in a manner to join the lips 22, 24 to one another and form a sealed joint therebetween.

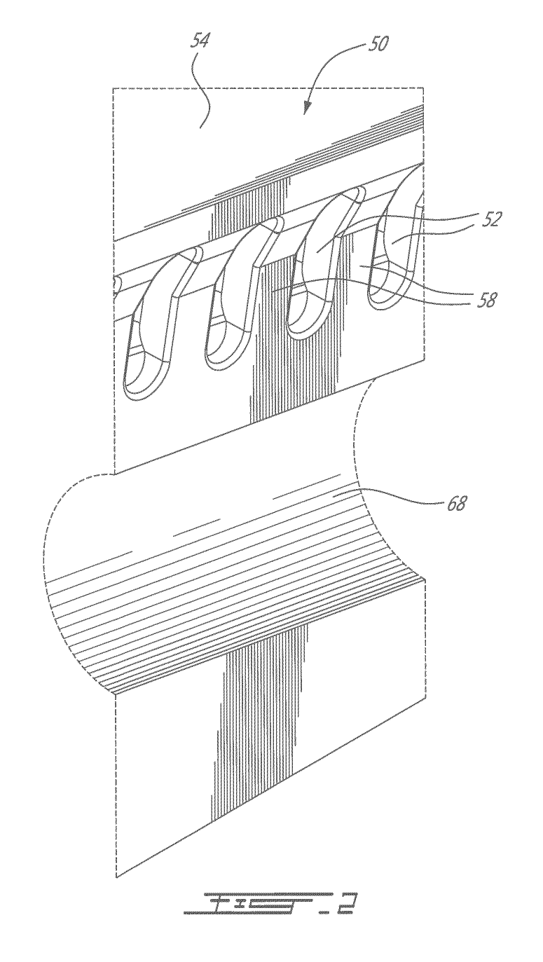

Indeed, as shown in FIG. 2, the ribs 38 can be formed by correspondingly shaped and configured voids in the mould 50, which will be referred to herein as rib negatives 52. Accordingly, the rib negatives 52 are regularly interspaced from one another in corresponding mould halves 54, 56, with FIG. 2 showing only one half 54 of the mould. The portions of the mould which are located between the rib negatives will be referred to herein as abutments 58.

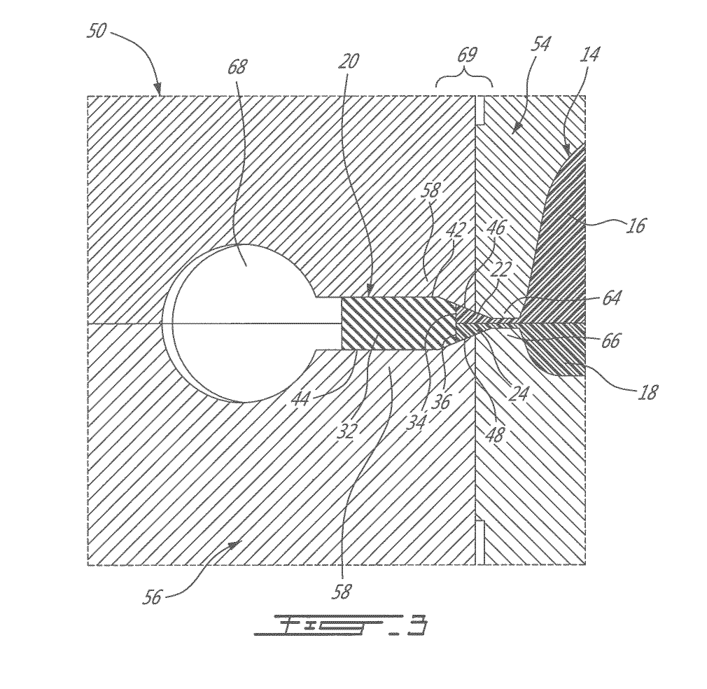

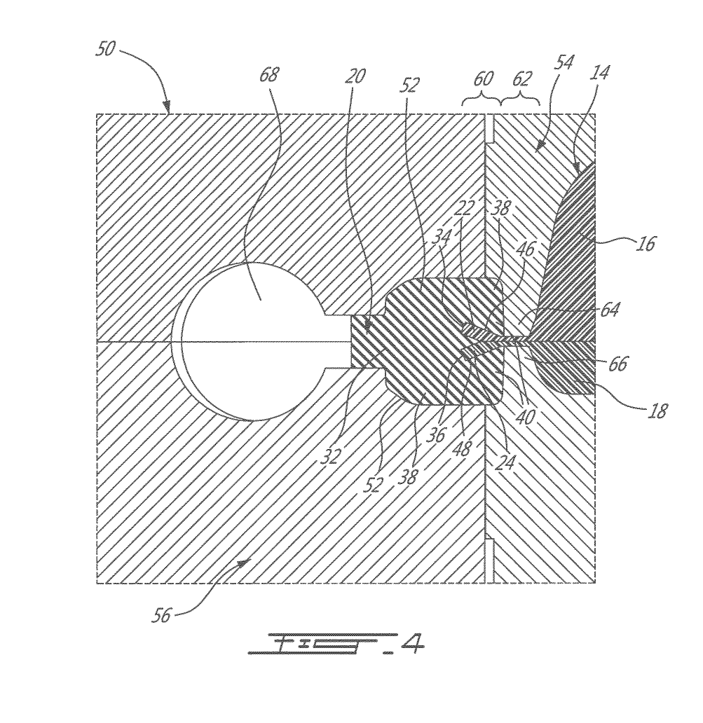

FIGS. 3 and 4 each show a cross-section showing the electronic device 11 trapped between the two mould halves 54, 56, with the overmoulded joint member 20. FIG. 3 is taken transversally across a pair of abutments 58, whereas FIG. 4 is taken transversally across a pair of rib negatives 52, shown here filled by the flange 32 and corresponding ribs 38 of the joint member 20.

As shown in FIG. 3, the pair of abutments 58 from corresponding halves 54, 56 of the mould 50 can be designed to compress the area of the superposed lips which is located between rib negatives during the step of overmoulding the joint member 20. The flange 32 can be seen extending transversally from the ends 34, 36 of the lips 22, 24.

FIG. 4 shows a cross-section taken along a pair of opposed rib negatives 52. As shown in FIG. 4, a portion 60 of the lips located in the rib negatives 52 is free from the compression of the mould 50 prior to feeding the elastomeric material of the joint member 20 into the mould 50. By comparing FIGS. 3 and 4, one can appreciate how the free portions 60 of the lips 22, 24 extend longitudinally between two adjacent abutments 52, and also transversally protrude from an inner portion 62 of the lips 22, 24 which is ‘nipped’ in compression between two features of the mould 50 which will be referred to herein as nips 64, 66. When the elastomeric material of the joint member 20 is fed in its liquid/viscous state, which can be done along a parallel channel 68 in this example, the surface tension of the elastomeric material under the clamping force of the mould as well as the injection pressure forces and stretches the free portions 60 of the lips 22, 24 apart from one another, but the freedom of movement of the free portions 60 of the lips 22, 24 is limited as adjacent portions of the lips 22, 24 are trapped in compression between the adjacent pairs of abutments 58 and between nips 64. Accordingly, tension is built into the free portions 60 of the lips 22, 24 due to the stretching, and this tension can eventually compensate for the penetrating pressure of the liquid/viscous elastomeric material. A satisfactory amount of the liquid/viscous elastomeric material can be allowed to form the pair of ribs 38 which covers the two opposite faces 46, 48 of the free portion 60 of the lips 22, 24 and, once solidified, join and seal the free portions 60 of the lips 22, 24 to one another. In practice, the channel 68 is also filled with elastomeric material after moulding and forms a trim which is removed after overmoulding. In FIGS. 3 and 4, the trim is not shown for simplicity.

Referring back to FIG. 1A, as the longitudinal spacing distance 70 between adjacent ribs 38 is limited, the degree of joining and sealing can be satisfactory even though the opposite faces 46, 48 of the lips 22, 24 are not covered by the joint member 20 between the ribs 38. Indeed, as shown in FIG. 3, between the ribs 38, at the location of the abutments 58, only the adjacent ends 34, 36 of the lips 22, 24 are covered by the flange 32 of the joint member 20 and the faces 46, 48 of the lips 22, 24 are uncovered. Moreover, the interspaced spacings between the ribs 38 along the joint member 20 can act as hinges and allow a greater amount of bending ability/transversal flexibility than if a pair of continuous rib elements extended continuously the periphery of the superposed lips.

Accordingly, the method of joining the lips can include: positioning the lips 22, 24 in a superposed manner in a mould 50 (e.g. such as shown in FIGS. 3 and 4); compressing the superposed lips 22, 24 in a plurality of compression locations (one of which is shown in FIG. 3) with corresponding pairs of abutments 58 of the mould which are interspaced from one another along the length of the lips 22, 24, the plurality of pairs of abutments 58 being interspaced from one another by corresponding pairs of rib negatives 52 (one of which is shown in FIG. 4); and overmoulding the joint member 20 onto the superposed lips 22, 24 in the mould, the joint member 20 having a flange 32 projecting from ends 34, 36 of the lips 22, 24, and a plurality of pairs of ribs 38 filling the rib negatives 52, each rib 38 of a corresponding pair protruding from the flange 32 and extending over a face 46, 48 of a corresponding one of the lips 22, 24.

Referring to FIG. 1B, concerning the press-switch system 30, the press-switch unit 28 has an actuator 72 which is resiliently movable along a pressing axis 74 for activation. A plate 76 is held within the housing 14, between the corresponding housing member 16 and the actuator 72, in a manner that while being trapped, it remains free to move along the pressing axis 74. When pressure is applied by a user on an external face 78 of the housing member 16, the pressure is transmitted to the plate 76 which, in turn, moves the actuator 72 along the pressing axis 74 and triggers the switch action. In the embodiment shown in FIG. 1B, the plate 76 is held by a corresponding holding feature, referred to as a holder 80 herein, which is formed in an inner face 82 of the housing member 16 and traps the plate 76 against a recessed portion 84 of the inner face 82. An annular recess 75 can also be provided around the holder and the thickness of the elastomeric material of the housing member can be limited by the annular recess 75 to allow freedom of movement of the elastomeric material and facilitate displacement of the plate 76 along the pressing axis 74. In an alternate embodiment, the plate can be slidingly received within a guide made integral to the electronic unit, for instance. It will be noted that, in this embodiment, a depression 86 is formed in the external face 78 of the housing member 16, in alignment with the pressing axis 74, to allow the user to easily feel the location of the switch by the touch, and can also prevent the user from inadvertently activate the switch. It will be understood that the press-switch system can be embodied in applications having a single housing member of elastomeric material covering the press-switch unit.

The method of joining housing members 16, 18 of elastomeric material as shown in FIG. 1A, and the press-switch system 30 shown in FIG. 1B, can be embodied alone, or together, in a wide variety of applications. One possible application which can combine both the method of joining housing members 16, 18 and the press-switch system 30 will now be described in a manner to provide an illustrative example application.

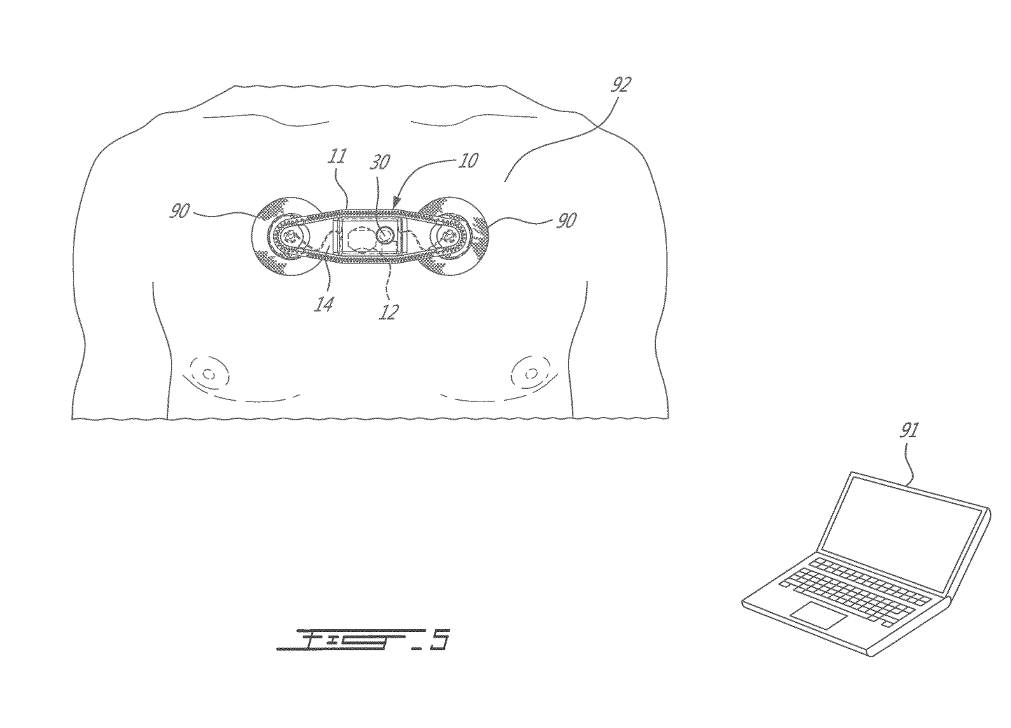

FIG. 5 shows an example of an electronic device 11 which embodies both the method of joining housing members 16, 18 and the press-switch system 30. In this example, the electronic device 11 is a wearable monitor 10, and more specifically an electrocardiogram (ECG) monitor which can be used to obtain an electric signal from medical electrode assemblies 90 adhered to the skin 92 of a patient's thorax.

Indeed, a wearable monitor 10 can require electronics to store or transmit the signal and create electrocardiogram data, which can later be accessed by a computer 91, for instance. When the wearable monitor 10 is designed to be used continuously, especially when used over several days or more, water-resistance can be required of the housing 14. In the case of ECG applications, the housing 14 can also require a significant amount of flexibility. Indeed, in particular situations, such as when the patient is sleeping on the side for instance, rigidity of the wearable monitor 10 can cause stress in the adhesive bond between the electrodes assemblies 90 and the patient's skin 92, which can cause noise in the signal, or even cause the wearable monitor 10 to disconnect from the electrode assemblies 90, or the electrode assemblies 90 to detach from the patient's skin 92, which is undesirable. For these reasons, elastomeric materials appear as an interesting choice for the housing 14. Moreover, in such applications, a press-switch system 30 can be used to allow the patient to add a marker in a corresponding temporal location in the data file containing the electrocardiogram data, such as to mark a moment in time when the patient feels a particular symptom, for instance. In this manner, the marker can later be used when accessing the electrocardiogram data to identify temporal locations which require a heightened level of attention, for instance. It will be understood that intuitiveness and ease of use can be particularly important in such applications.

In this example, the wearable monitor 10 can be said to be of the integrated connector-type, that is, of the type of wearable monitor which does not use wires to connect to the electrodes, but rather electrode connectors directly integrated within an elongated, bandage-like housing having the electronic unit integrated therein. More specifically, the housing has an electrode connector at each end of its length. The housing can thus preferably have a significant amount of flexibility and water-resistance.

In this specific embodiment, the thermoformable elastomer (TPE) used was product number CL-40 manufactured under the trademark POLYONE by Versaflex. As shown in FIG. 3, in this embodiment, a sloping portion 69 of the abutments 58 connects the nips 64, 66, and the nips 64, 66 compress the lips 22, 24 by more than 50% of their initial thickness, preferably down to 25-35% of their initial thickness, in this embodiment. This configuration can lead to a satisfactory embodiment for at least the illustrated application.

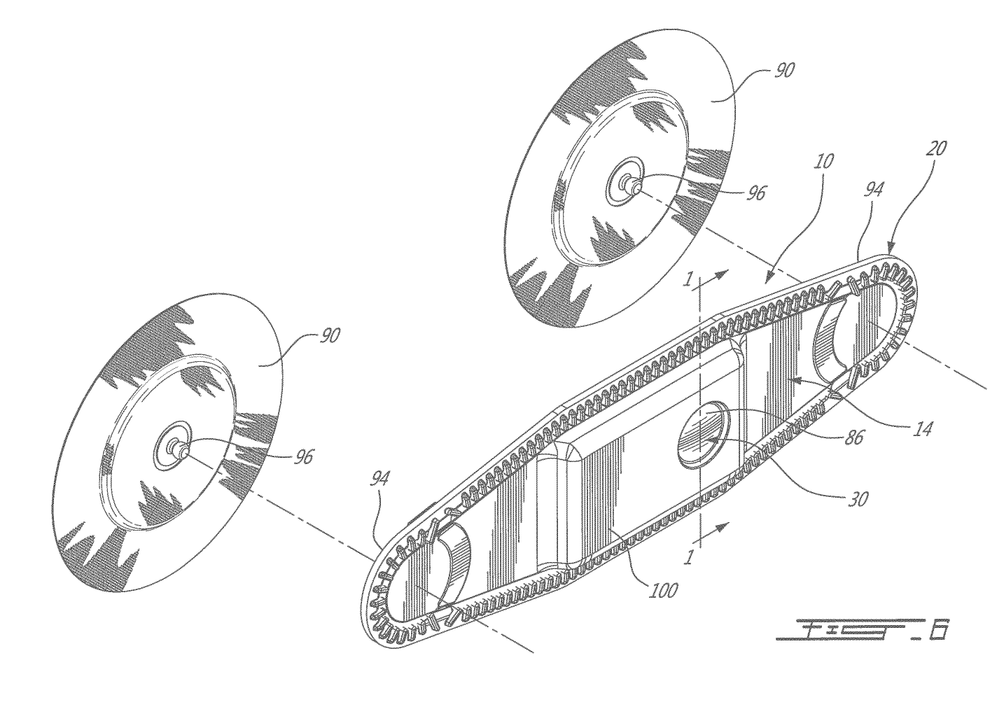

FIG. 6 shows the wearable monitor 10 without the patient, with the medical electrode assemblies 90 being disconnected. The wearable monitor 10 is elongated and has electrode connectors 94 at each end, which are designed to mate with corresponding connectors 96 of the medical electrode assemblies 90. The connectors between the medical electrode assemblies 90 and the monitoring unit 10 are push-button type connectors in this case, which can offer both mechanical and electrical connection. Any other suitable connector configuration can be used in alternate embodiments. Moreover, it will be noted that the medical electrode assemblies 90 shown in the figure have an optional adhesive extender covering an ‘off the shelf’ electrode unit. This configuration is used purely for exemplary purposes and any suitable medical electrode assembly can be used in alternate embodiments. The electronic unit 12 of the wearable monitor 10 is located within a cavity 98 formed between two superposed housing members 16, 18 which are joined to one another by a joint member 20. A central protuberance 100 on the outer one of the housing members 16 coincides with the location of the electronic unit 12, and bears a depression 86 which can be used by the user to locate the press-switch unit 28. FIGS. 1A and 1B are taken along cross-section line 1-1 of this example wearable monitor 10.

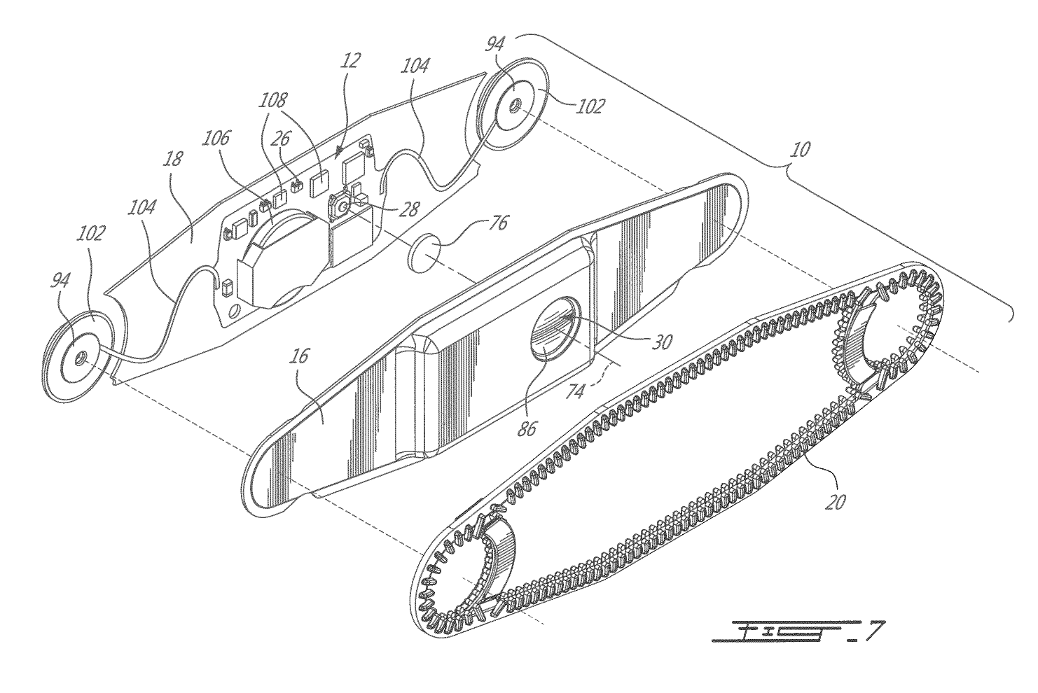

FIG. 7 shows the housing members 16, 18 of the wearable monitor 10, and the joint member 20, exploded. The housing members 16, 18 can be seen as they are prior to overmoulding the joint member 20. The joint member 20 is designed to be formed only in its overmoulded state, and is not normally seen in a standalone manner as shown in this figure for illustrative purposes. The electrode connectors 94 in this example are female press-button connectors assembled to annular sheets 102 of elastomeric material and are initially separate from the inner one of the housing members 18. The electrode connectors 94 are connected to the electronic unit 26 by wires 104. The electronic unit 26 receives a battery 106, circuit components 108, and the press-switch unit 28 on an electronic board 26, the electronics board 26 itself being received on the housing member 18. When the components are positioned into the mould 50 for overmoulding of the joint member 20, the electrode connectors 94 can be received in and engaged with corresponding features of the mould 50. In alternate embodiments, the type, quantity and diversity of electronic components can vary.

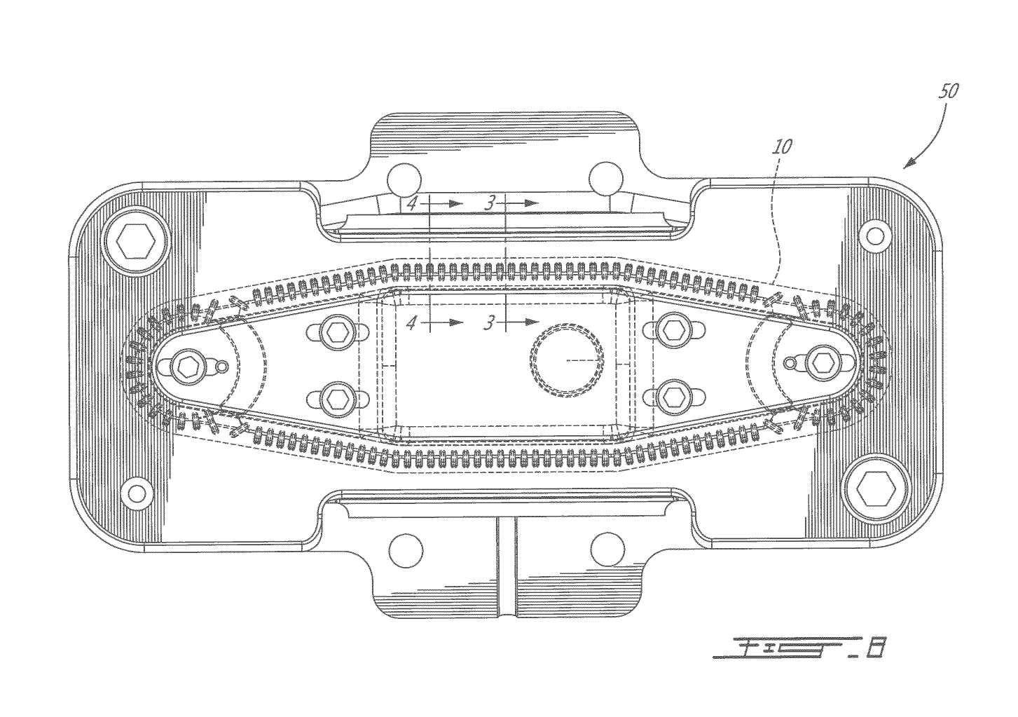

FIG. 8 shows the mould 50 assembled with the wearable monitor 10 trapped therein after overmoulding, the cross-sectional views of FIGS. 3 and 4 being taken from this example, along corresponding cross-sections lines 3-3 and 4-4. The mould 50 can have a suitable internal construction with inserts and a ramification of channels, such as known in the art.

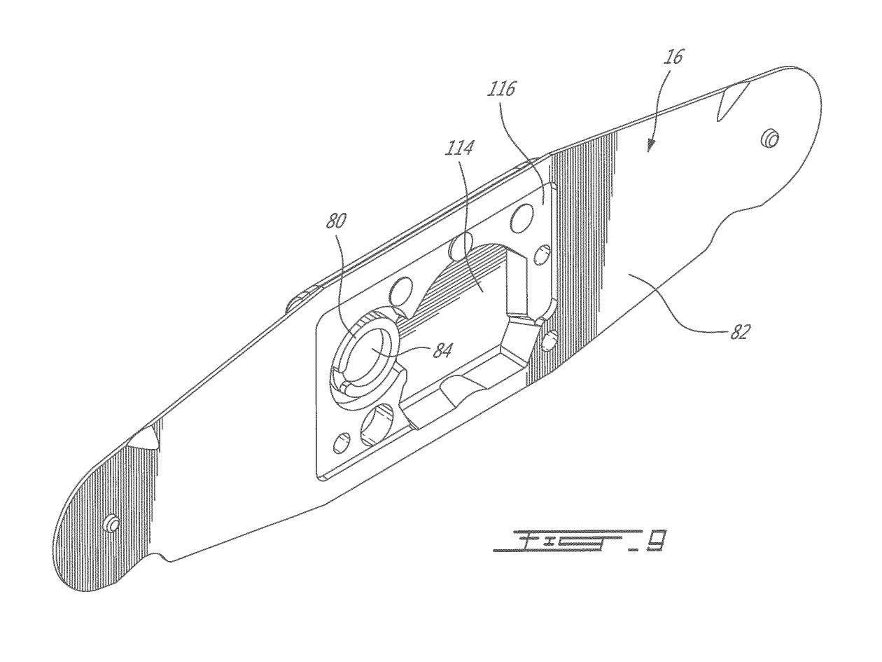

FIG. 9 shows the inner face 82 of the outer one of the housing members 16 prior to overmoulding. The holder 80 can be seen to be provided in the form of a quasi-annular protrusion extending from the inner face 82 of the housing member 16, delimiting a plate recess 84 within which the plate 76 can be snugly received and held against the inner face 82 of the housing member 76. The outer one of the housing members 16 also has a battery recess 114 within the cavity 116, into which the battery 106 of the electronic unit 12 can extend during overmoulding and later use.

It will be understood that methods of joining lips of elastomeric members and press-switch systems based on the teachings of this specification can be embodied, in combination or individually, in other embodiments than the wearable monitor shown and described herein. For instance, applications where an electronic circuit needs to be protected from dust or water in a flexible enclosure and when the application is misadapted to direct overmoulding such as can be the case in the of unsealed components such as a mechanical switch or a battery.

As can be understood, the examples described above and illustrated are intended to be exemplary only. The scope is indicated by the appended claims.

1. An electronic device comprising an electronic unit housed within a housing, said housing comprising two housing members and a joint member each being made of an elastomeric material, the two housing members having superposed lips extending at least partially along a periphery thereof, the joint member having a flange projecting from ends of the lips, and a plurality of pairs of ribs, the pairs of ribs being interspaced from one another along the superposed lips, and each rib of each pair protruding from the flange and extending over a face of a corresponding one of the lips.

2. The electronic device of claim 1 wherein the ribs are elongated, and the pairs of ribs are interspaced from one another by a spacing distance having about the width of the ribs.

3. The electronic device of claim 1 wherein the electronic unit is housed within an air-filled cavity within the housing, and further has a battery.

4. The electronic device of claim 3 wherein the electronic unit further has a press-switch unit.

5. The electronic device of claim 1 wherein the flange has sloping faces extending from corresponding faces of corresponding ones of the lips, and leading to parallel faces being oriented parallel to the corresponding faces of corresponding ones of the lips.

6. The electronic device of claim 1 being provided in the form of a wearable monitor having a monitoring circuit sealingly housed within the housing, the housing being flexible and having two opposite ends each bearing a corresponding electrode connector, the monitoring unit being connected to both electrode connectors.

7. A method of joining lips of an elastomeric material, the method comprising:
positioning the two lips in a superposed manner in a mould, the mould compressing the superposed lips in a plurality of abutments interspaced from one another along the length of the lips, the plurality of abutments being interspaced from one another by corresponding rib negatives;
overmoulding a joint member onto the superposed lips in the mould, the joint having a flange projecting from ends of the lips, and a plurality of pairs of ribs filling the rib negatives, each rib of a corresponding pair protruding from the flange and extending over a face of a corresponding one of the lips.

8. The method of claim 7 further comprising, during said overmoulding, compressing a nipped portion of the superposed lips continuously along at least a portion of the length of the lips, wherein the abutments have narrowing sloping faces leading to the nipped portions and extending partially along the flange portion and partially along the superposed lips after said overmoulding.

9. The method of claim 7 wherein said overmoulding includes sealing an electronic unit within an air-filled cavity formed within elastomeric material.

10.-15. (canceled)
